(19) United States
(12) Patent Application Publication
(10) Pub. No.: US 2005/0240197 A1
(43) Pub. Date: Oct. 27, 2005

(54) Device and method for inserting, positioning and removing an implant

(76) Inventor: Stanley J. Kmiec JR., Morgantown, PA (US)

Correspondence Address: JONES DAY, 222 EAST 41ST STREET, NEW YORK, NY, 10017-6702, US

(21) Appl. No.: 10/831,569

(22) Filed: Apr. 23, 2004

(51) Int. Cl. A61B 17/58

(52) U.S. Cl. 606/100

(57) ABSTRACT

A device for inserting an implant into a bone and/or backing-out/removing an implant from a bone. The device comprises a shaft, a head connected to the shaft, a channel through the head, and a locking mechanism. A handle may also be provided on the shaft. In one position, the locking mechanism engages the head, thereby preventing the head from pivoting and/or rotating relative to the shaft. In another position, the locking mechanism is disengaged from the head, such that the head can pivot and/or rotate relative to the shaft. The device may be used with a inserter. The locking mechanism may have means for holding a portion of the inserter in the channel. A method of inserting an implant into a bone and/or backing-out/removing an implant from a bone is also provided.

### FIELD OF THE INVENTION

The present invention is directed to a device and method for inserting, positioning and/or removing an implant from bone and, in particular, a device having a portion which can be selectively fixed or freely pivotable and/or rotatable with respect to a body portion of the device.

### BACKGROUND OF THE INVENTION

Intramedullary rods or nails can be used for internal fixation and reduction of a fractured bone, such as for example a long bone. The intramedullary rod is generally inserted into the marrow canal of a long bone, such as the femur, and across the fracture so that the fracture portions are properly aligned in close apposition. The rod is typically inserted in one end of the long bone and extends into the shaft of the bone. Moreover, the rod may be designed to be backed-out of the bone to adjust the rod, remove the rod, or pull two halves of a fracture together. Various devices can aid in the insertion and/or backing-out and/or removal of the intramedullary rod.

The implant insertion and positioning device chosen by a surgeon depends on the technique the surgeon desires to use, the desired results, the implant, and parameters of the operation. If a surgeon desires to freely hammer an implant into a bone and the surgical procedure so allows, a surgeon can use a hammer-type insertion device having a head and a shaft. The head on such devices has an orientation which is fixed with respect to the shaft. A rod inserter having an enlarged portion is attached to the intramedullary rod. The surgeon repeatedly strikes the enlarged portion of the rod inserter with the head of the hammer-type insertion device to drive the rod into the marrow canal.

On the other hand, if a surgeon desires or the surgical procedure so requires and/or allows, the surgeon can use a hammer-type insertion device where the head pivots about the shaft. This hammer-type insertion device may be used with a rod inserter, which is attached to the rod and consists of an elongated shaft having proximal and distal ends. The shaft may also have an enlarged portion at its proximal end, which is closest to the surgeon, and/or an enlarged portion at its distal end, the end closest to, and typically attached to the intramedullary rod. The hammer-type device is positioned on the shaft and travels along the shaft as it undergoes a reciprocating motion that allows a surgeon to repeatedly strike either enlarged portion. A pivotable head allows for ease of movement as the device is reciprocated along the shaft. To drive a rod into the bone, the surgeon strikes the enlarged portion at the distal end of the shaft with the head of the hammer-type device. To back-out or remove a rod from the bone, an operator strikes the enlarged portion at the proximal end of the shaft.

### SUMMARY OF THE INVENTION

The present invention relates to a device for impacting or inserting an implant into bone, and/or backing-out, extracting or removing the implant from the bone, and/or repositioning an implant in bone. In particular, the device can be used to insert a rod into a bone, back-out and/or remove a rod from a bone, or reposition a rod in a bone. The device comprises a shaft, a head, optionally a channel through the head, and a locking mechanism. The locking mechanism may engage the head so that the head does not pivot and/or rotate relative to the shaft. In addition, the locking mechanism may be disengaged from the head, thereby enabling the head to pivot and/or rotate freely with respect to the shaft. Preferably, the locking mechanism is associated with the shaft of the device. The device may also have means for retaining a guide in the optional channel. Additionally, the locking mechanism may have means for tightening the locking mechanism on the shaft.

The present invention also relates to methods for impacting or inserting an implant into bone, backing-out, extracting, or removing an implant from a bone, and/or repositioning an implant in a bone. In one method of impacting, repositioning or backing-out a rod from a bone, the device may be used with an implant inserter which is attached to the rod. The inserter may have an aiming arm and an enlarged portion proximate its end. One exemplary method of using the device includes the steps of: moving the locking mechanism so that it engages the head, thereby fixing the head relative to the shaft (i.e., the head does not pivot, rotate, or translate relative to the shaft); and striking the rod or the enlarged portion of the inserter (if an inserter is used) with the head of the device.

Another alternative method of using the device involves the steps of: attaching an implant inserter, which comprises a guide to an implant (such as, for example, an intramedullary rod); adjusting the locking mechanism so that it is disengaged from the head, thereby allowing the head to pivot or rotate relative to the shaft; positioning the device onto the guide; and reciprocating the device along the guide. The inserter used with this method may have an enlarged portion at one end of the guide proximate the rod and/or an enlarged portion at the other end of the guide. An operator can strike the rod or the enlarged portion proximate the rod to insert the rod into the bone. Alternatively, an operator can strike the enlarged portion proximate an operator to back-out, remove or reposition the rod from the bone.

In another embodiment, the head of the device may also contain a slot, which communicates with the channel of the head, preferably intersecting the channel at an angle, most preferably perpendicular to the channel. The head can be positioned on a guide by moving the slot of the head onto the guide shaft until the shaft reaches the channel. The device is then rotated to align the longitudinal axis of the guide shaft with the longitudinal axis of the channel. In yet another embodiment, the head may have a helical slot, which intersects the channel of the head. The helical slot is inserted onto the guide shaft and, as the helical slot is advanced onto the guide shaft, the head threads, rotates or twists until the longitudinal axis of the guide is aligned with the longitudinal axis of the channel.

### BRIEF DESCRIPTION OF THE DRAWINGS

The present invention can be better understood by reference to the following drawings, wherein like references numerals represent like elements. The drawings are merely exemplary to illustrate certain features that may be used singularly or in combination with other features and the present invention should not be limited to the embodiments shown.

FIG. 1 is a perspective view of the implant insertion, removal, positioning device according to the present invention;

FIG. 2 is a side view of the device as shown in FIG. 1;

FIG. 3 is a rear view of an alternative embodiment of the device according to the present invention;

FIG. 4 is a cross-sectional view of the head of the device as shown in FIGS. 1 and 2;

FIG. 4A is a cross-sectional view of a portion of an exemplary holding member;

FIG. 4B is a cross-sectional view of a portion of another exemplary holding member;

FIG. 4C is a cross-sectional view of a portion of another exemplary holding member;

FIG. 4D is a cross-sectional view of an alternative embodiment of the head of the device as shown in FIGS. 1 and 2;

FIG. 4E is a perspective view of the head of the device as shown in FIGS. 1 and 2 with an alternative exemplary holding member;

FIG. 5 is a perspective view of an alternative embodiment of the device according to the present invention;

FIG. 6 is a perspective view of an alternative embodiment of the head of the device according to the present invention;

FIG. 7 illustrates a method of using the device as shown in FIG. 1; and

FIG. 8 illustrates an alternative method of using the device as shown in FIG. 1.

### DETAILED DESCRIPTION

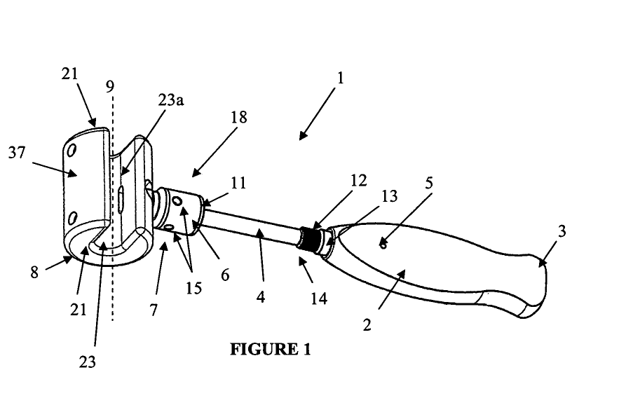

FIG. 1 shows an exemplary embodiment of tool 1 for use in inserting, backing-out, extracting, removing and/or positioning an implant. It should, however, be understood that those of ordinary skill in the art will recognize many modifications and substitutions which may be made to various elements of the present invention. Tool 1 can be used by an operator to insert an implant into, back-out or remove an implant from, and/or position or reposition an implant in a bone. For example, tool 1 may be used to insert an intermedullary rod or nail into or back-out/remove a rod from the medullary canal of a bone. As illustrated in FIG. 1, exemplary tool 1 may include a handle 2, a shaft 4, a locking mechanism 7 (including a locking member 6), and an impacting body or head 8 pivotally connected to shaft 4. It should be noted that the term pivot and rotate is used interchangeably herein.

Handle 2 permits an operator to grab and hold tool 1 during use. Handle 2 is preferably ergonomically contoured for use by the operator and may contain grip enhancing surfaces, materials and/or projections. It should be noted that handle 2 is not necessary to the functioning of tool 1 since an operator can grab shaft 4 instead. Handle 2 may be made of plastic, rubber, metal, wood, a composite material (i.e., made of at least two materials), or any other suitable material. For example, handle 2 may be made of an impregnated linen or a phenolic resin. Alternatively, handle 2 can be made from a molded polymer (e.g., Delrin®). Handle 2 may also have a core material surrounded by another material. For instance, an aluminum core may be surrounding by a silicon jacket. Various factors can be considered when determining the material used to make handle 2, including ability to withstand sterilization/cleaning (e.g., using an autoclave; cleaning products used for sterilization in hospitals), feel (e.g., a molded material gives a softer feel in a operator's hand), weight, durability (e.g., ability to withstand impact/load forces, ability to be dropped), resistance to staining (e.g., from blood or substances used in surgery or to clean tool 1), the ability to withstand heat, and the ability to grip the handle, particularly with latex gloves which are generally used during surgery. Other factors, not listed, may also be relevant.

The handle 2 can be any shape or size. For example, it may be desirable for handle 2 to be shaped to fit the contours of a hand (i.e., ergonomic). Moreover, the end 3 of handle 2 can be flared to prevent handle 2 from slipping from an operator's hand during use. Additionally, to allow for enhanced gripping of handle 2 by an operator, handle 2 may have a grip (not shown), which covers or is positioned in a least a portion of handle 2.

The grip may be made out of the same materials as handle 2. The grip may have indents or protrusions. And, the factors used to select the material for handle 2 are also applicable to choosing material(s) for the grip. The grip may be a separate piece from handle 2, for example, the grip may be positioned as an inset (not shown) in the handle. The grip may also be over-molded (i. e., formed by molding a material over handle 2). A grip made of rubber may be used. Alternatively, the grip may be integral or monolithic with handle 2 (i.e., the handle and grip are made of one piece of material). In such a configuration, the grip portion of handle 2 can be textured.

Shaft 4 can be attached to handle 2 or be one integral piece with handle 2. Shaft 4 can be made from, for example, a hardened material such as stainless steel. However, shaft 4 may be made of any suitable material. Various factors may be used to determine the material of shaft 4, including ability to withstand sterilization/cleaning, weight, durability, resistance to staining, and heat treatable. Other factors, not listed, may also be relevant. A stainless steel shaft 4 can have a diameter between about approximately 5 mm and about approximately 20 mm, preferably between about approximately 6 mm and about approximately 15 mm, and most preferably between about approximately 7 mm and about approximately 10 mm. It should be understood that the diameter of shaft 4 depends on the material used and can be any diameter so long as the diameter of shaft 4 is sufficient enough so that shaft 4 will not break or bend from repetitive use of tool 1. Moreover, shaft 4 may have a length measured from handle 2 to head 8 between about approximately 20 mm and about approximately 150 mm, preferably between about approximately 50 mm and about approximately 140 mm, and most preferably between about approximately 100 mm and about approximately 130 mm. The length of shaft 4 is one factor, amongst others, enabling an operator to properly swing tool 1, and ensuring proper leverage and momentum of tool 1 for hammering. Furthermore, as shown in FIG. 1, shaft 4 can be straight, however, any other shape (e.g., S-shape, L-shape, curved), which allows an operator to hammer an implant and enables the proper functioning of the locking mechanism 7 is envisioned.

Shaft 4 may abut handle 2 or extend through a portion or the entire length of handle 2. Shaft 4 can be attached to handle 2 in numerous ways, including friction fitting, welding, chemical or molecular bonding, or gluing. In another embodiment, shaft 4 can have an external thread (not shown) engaging an internal thread (not shown) of handle 2. In an alternative embodiment, handle 2 may have an opening 5 which receives a pin, screw, set screw, rod, bar or other retaining means which attaches shaft 4 to lock handle 2 and shaft 4 relative to each other.

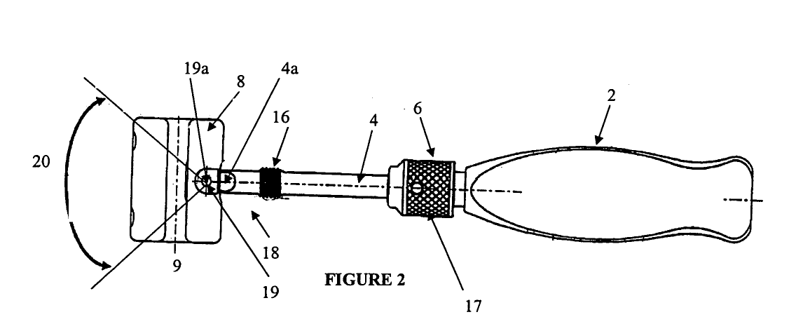

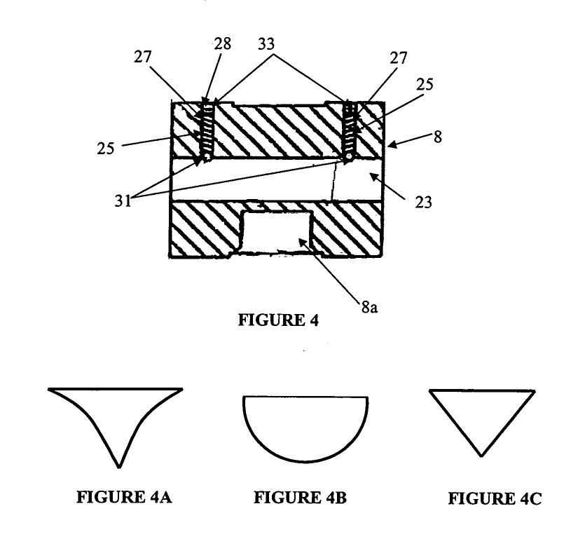

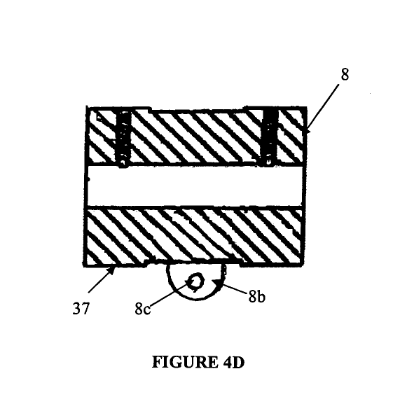

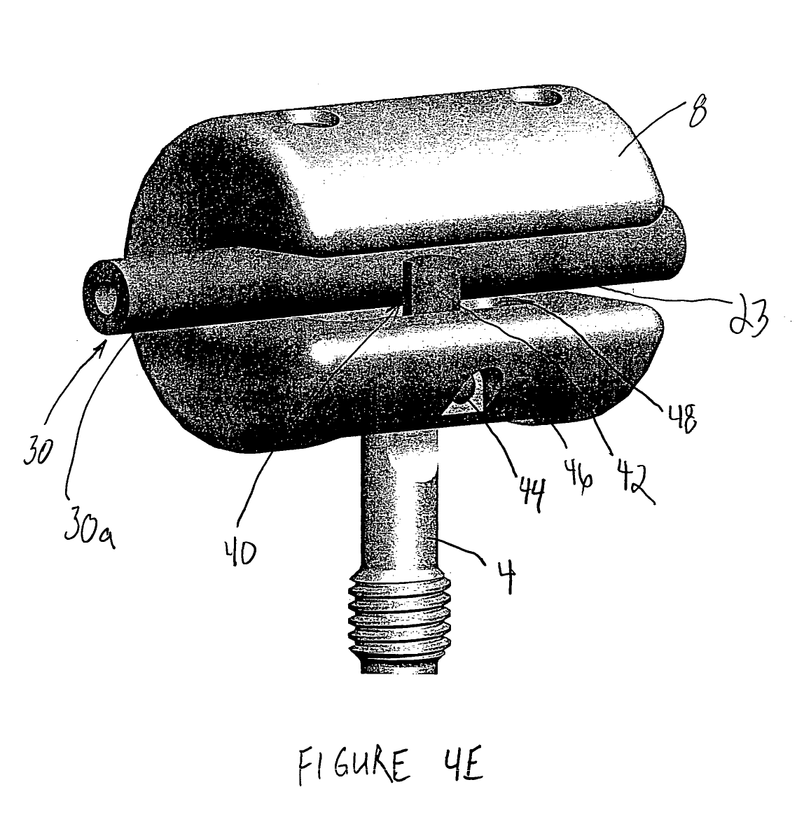

Head 8 is connected to shaft 4. Head 8 may be preferably pivotally and/or rotationally connected to shaft 4. In one embodiment, as shown in FIG. 2, a portion 4a of distal end 18 of shaft 4 may be flattened or have a reduced diameter with a hole 19 therethrough. Reduced shaft portion 4a may be inserted into a open area 8a (FIG. 4) formed in the head 8. A pin 19a can be positioned through hole 19 such that shaft 4 pivots or rotates about pin 19a. The pin 19a can be fixed with respect to head 8 by, for example, force/press fitting, welding, chemically or molecularly bonding, or gluing into receiving portion(s) (not shown) of head 8. Alternatively, a pin can be fixed to shaft 4 (or shaft 4 may have protrusions (not shown) extending from opposite side of shaft 4). The pin or protrusions can be positioned in receiving portion(s) (not shown) of head 8 such that head 8 can pivot or rotate with respect to shaft 4. In this manner head 8 can pivot about shaft 4.

It should be understood that open area 8a is not required. For example, head 8 may have an extension 8b (FIG. 4D) (one or more extensions 8b may be used) extending from outer surface 37 of head 8. The extension 8b can have hole 8c to receive a pin 19a. The hole 19 in shaft 4 can be aligned with hole 8c in extensions 8b. Pin 19a may be inserted through hole 8c in extension 8b and hole 19 in shaft 4. In such a configuration, the angle 20 through which shaft 4 can swing or pivot (hereinafter referred to as the “angle of pivot”) is constrained by shaft 4 contacting head 8.

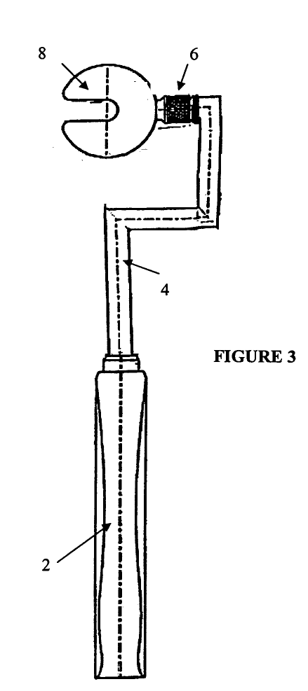

Shaft 4 and head 8 can be configured so that head 8 has an angle 20 of pivot of about 360° (e.g., where the shaft 4 is attached to the side of head 8 (FIG. 3)) but more preferably the angle 20 of pivot can be between about approximately 30° and about approximately 270° degrees, and even more preferably between about approximately 30° and about approximately 180° degrees, and most preferably between about approximately 30° and about approximately 90° degrees. The angle 20 through which shaft 4 pivots with respect to the head 8 depends upon the clearance of shaft 4 in open area 8a in the head 8. The angle 20 of pivot preferably would allow for ergonomic swinging of tool 1. Some factors which contribute to determining an appropriate angle 20 of pivot include the length of the shaft/handle and the amount of throw that is required to generate the energy necessary for inserting an implant.

In another embodiment, shaft 4 may have a ball (not shown) at distal end 18 and head 8 may have a socket (not shown) to receive the ball of shaft 4. Alternatively, shaft 4 and head 8 may be connected by a swivel joint (not shown) so that head 8 can be rotated about shaft 4. Any other means of connecting shaft 4 and head 8 can also be used in a manner so that head 8 can pivot and/or rotate with respect to shaft 4 in at least one direction or about at least one axis.

A locking mechanism 7 may be provided to fix shaft 4 and head 8 with respect to each other, thereby preventing pivotal or rotational movement of head 8 about shaft 4. It should be understood that a locking mechanism includes any mechanism which functions to lock shaft 4 and head 8 relative to each other and which also allows the shaft 4 to be unlocked so that shaft 4 and head 8 can pivot with respect to each other. The locking mechanism may be associated with, or may be attached to shaft 4, or may be attachable to shaft 4 (i.e., capable of being attached/detached from shaft 4). The locking mechanism can consist of a single piece or more than one piece.

FIGS. 1 and 2 illustrate an exemplary locking mechanism 7. The locking mechanism of FIGS. 1 and 2 includes a locking member 6 disposed about shaft 4 (FIGS. 1 and 2). Locking member 6 can be made of a hardened material, such as stainless steel, however, other materials can also be used. The same factors that are used to determine the material for shaft 4 can also be used to determine the material, shape, and dimensions of locking member 6. Other factors may also be considered such as the ability to be tightened on shaft 4 and resistance to deformation caused by impact loads from head 8. To enable tightening of locking member 6 on shaft 4, member 6 should be shaped and of sufficient size to allow an operator to grip locking member 6 with his/her fingers or, as discussed in greater detail below, with a tool or to position a tool into locking member 6. It should be understood that locking member 6 can be any shape, size, or thickness so long as it can perform its function of keeping shaft 4 and head 8 fixed relative to each other. In one exemplary embodiment, not shown, locking member 6 can be a nut.

It should be noted that the portion of locking member 6 that engages the handle 2 or head 8 can be configured to assist in holding the locking member 6 against the handle 2 or help in fixing the head 8 relative to shaft 4. For example, locking member 6 can have a flat portion for engaging a corresponding flat portion of handle 2 and/or head 8. Locking member 6 may also have a textured portion, for example teeth, which engages a corresponding textured or smooth portion, for example teeth, on handle 2 and/or head 8.

Locking member 6 may have internal threads (not shown) to engage proximal threads 12 or distal threads 16. Threads 16 may be located proximate head 8 and function to hold locking member 6 against head 8 at distal end 18 of shaft 4. Threads 12 function to hold locking member 6 at a position on shaft 4 such that locking member 6 does not engage head 8. In FIG. 1, threads 12 are located proximate handle 2 such that when locking member 6 engages threads 12, a portion 11 of locking member 6 engages enlarged diameter portion 13 and/or handle 2. This configuration allows for locking member 6 to be tightly fastened to the proximal end 14 of shaft 4.

By threading locking member 6 on proximal threads 12 or by tightly fastening locking member 6 against an enlarged portion, locking member 6 is restrained from moving as the operator uses the insertion, positioning, removing tool 1. More specifically, locking member 6 will not move along shaft 4 as the device is swung by an operator. Locking member 6 can be tighten on shaft 4 by manually twisting member 6 on threads 12 or 16 (i.e., an operator uses his/her fingers/hand). Member 6 may also have a grip (e.g., diamond knurl 17 as shown in FIG. 2) which is integral with member 6 and can take various forms, including a rough surface, grooves, or depressions on a portion or around the entire periphery of locking member 6. In another embodiment, locking member 6 can have a grip made from a separate piece of material. A grip can be positioned around the outer surface of member 6 or can be inlaid into member 6. For example, locking member 6 may be over-molded with a rubber jacket. Over-molded material may be made from any material that provides a better gripping surface for an operator (before, during, or after the operation) than a smooth surface.

In addition, to allow for further tightening, locking member may 6 have one or more opening 15, which can be any shape or size, positioned about the periphery of member 6. A device such as a spanner or pin wrench, rod, or bar (not shown) can be positioned in opening 15 and used to twist locking member 6 on shaft 4. The spanner or pin wrench, rod, bar, or other device provides an operator with leverage to further tighten locking member 6 on threads 12, 16. It should be understood that locking member 6 may be tightened only manually or, alternatively, there can be no manual tightening and the entire tightening step can be performed using a device such as a spanner or pin wrench, rod, bar or other device (not shown), or the member 6 can be manually tightened by the operator turning the member 6 and then further tightened using a tightening tool. In addition to or in place of using devices such as the spanner or pin wrench, rod, bar, or other device, an operator may use, for example, a plier, wrench, or similar device to grab the outer surface of locking member 6 and twist member 6 on threads 12 and 16. Moreover, to prevent member 6 from loosening on threads 12 and 16, a screw, set screw, bolt, pin, or other retaining member can be positioned to extend through member 6 and contact the surface and/or extend into threads 12, 16 and/or shaft 4.

In an alterative embodiment, threads 12 may be located at any location along the length of shaft 4. To lock locking member 6 tightly to shaft 4, a portion of threads 12 may be deformed and/or enlarged portion 13 may be positioned adjacent threads 14. In yet another embodiment, there may be only one threaded portion on shaft 4. The threaded portion preferably would cover enough of shaft 4 so that locking member 6 can be moved from a position adjacent head 8 to a position where member 6 disengages head 8. In still another embodiment, the entire shaft 4 may be threaded. In another embodiment, the shaft 4 may taper from a smaller diameter at distal end 18 to a larger diameter at proximal end 14 or have an enlarged portion. Locking member 6 can be force fitted over a portion of the taper or enlarged portion to hold it on shaft 4 at a sufficient distance from head 8 so that head 8 can pivot about shaft 4. Also, in any embodiment, locking member 6, in its disengaged position, may be moveable along shaft 4 (i.e., not locked to shaft 4) or may be designed to be removed all together from shaft 4 upon disengagement from head 8. The locking mechanism 7 may not comprise any threads at all.

In an alternative embodiment, a pin, screw, set screw, bolt, or other retaining means, can extend through member 6 and into or through shaft 4. The pin, screw, set screw, bolt or other retaining means may extend through opening 15 or some other opening that forms a through hole. The through hole may be threaded to engage corresponding threads on the pin, screw, set screw, bolt, or other retaining means. This configuration can be used to hold member 6 on shaft 4 anywhere along the length of shaft 4.

In yet another alternative embodiment, the locking mechanism may incorporate a bayonet fitting. For example, there can be one or more protrusions (not shown) around the periphery of shaft 4. The protrusion(s) may be a single prong, or may extend a portion or the entire length of shaft 4. Locking member 6 of this embodiment may have internal groove(s) along its length to receive the protrusion(s). Such a configuration allows for axial, but not rotational, movement of member 6 on shaft 4 where member 6 engage the protrusion(s). A pin, screw, set screw, bolt or other retaining means may be used to hold member 6 against head 8 or at any position along the length of shaft 4. Alternatively, a spring (not shown) can be positioned around shaft 4 and bias locking member 6 towards head 8. It should be appreciated that any combination of the embodiments and features disclosed herein can be used as a locking mechanism.

In an alternative embodiment, there may be no locking member 6. Such an embodiment may use a pin, set screw, screw, rod, bar, bolt or other means to fix shaft 4 so that shaft 4 does not pivot with respect to head 8. One of ordinary skill in the art can readily appreciate that other means of temporarily fixing shaft 4 and head 8 relative to each other is envisioned by the present invention.

As shown in FIG. 3, in an alternative embodiment, shaft 4 may be connected on the side of head 8. Shaft 4 may be shaped similar to a question mark and have a handle 2. The connection of shaft 4 and head 8 should allow for pivotal and/or rotational movement of head 8 relative to shaft 4. It should be noted, however, that shaft 4 can be connected anywhere on the outer surface 37 (FIG. 1) of head 8 preferably so that head 8 can pivot and/or rotate with respect to shaft 4, and tool 1 can be used to hammer an implant into, reposition, or remove/back-out an implant from a bone.

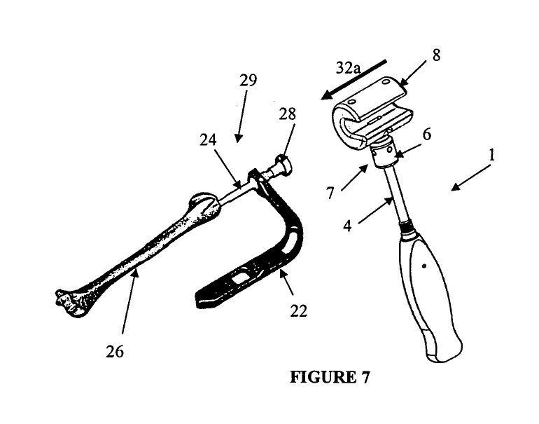

Head 8 can be any shape or size so long as head 8 can function to impact/strike an implant and/or portion of an insertion device, such as inserter 29 (FIG. 7) in order to impart a force to insert the implant into, reposition, or back-out the implant from a bone. Any portion of outer surface 37, but preferably faces 21,21a of head 8, can be used for impacting/striking an implant or insertion device. Additionally, faces 21, 21a of head 8 may have a piece of material attached thereto which is made of a stronger material than head 8 (e.g., a material which is more resistant to dents and/or deformation). For example, a piece of titanium may be welded to face 21 and/or 21a. In another embodiment, a softer material may be attached to faces 21, 21a to absorb the impact force from hammering an implant. For instance, a piece of rubber can be bonded to face 21 and/or 21a. It should be noted that a separate piece of material can be attached to any portion of head 8 that may contact another surface during hammering.

Head 8 may be made of stainless steel, but other materials can also be used. Any of the various factors discussed above with regard to choosing a material for shaft 4 or member 6 can be used to determine the material of head 8. In addition, other factors that may be considered is resistance to dents from hammering and impact strength. Moreover, head 8 can have a length between about approximately 30 mm and about approximately 150 mm, preferably between about approximately 40 mm and about approximately 100 mm, and most preferably between about approximately 50 mm and about approximately 75 mm. And, head 8 can have a diameter of between about approximately 30 mm and about approximately 100 mm, preferably between about approximately 35 mm and about approximately 75 mm, and most preferably between about approximately 38 mm and about approximately 60 mm. Various factors can be considered when determining the dimensions of head 8, including the weight and/or mass of the head 8 which is necessary for generating the energy needed to insert an implant.

Head 8 may also have a channel 23 and a slot 23a intersecting the channel 23. Head 8 and channel 23 may share a longitudinal axis 9, however, head 8 and channel 23 may each have a separate axis, which can be parallel or at any angle with respect to each other. Slot 23a communicates with channel 23 and outer surface 37 of head 8, thereby providing a path for inserting a guide 30, such as longitudinal member 30a shown in FIG. 7, into channel 23. Slot 23a may run the length of head 8 and intersect channel 23 along its entire length as shown in FIG. 1. Channel 23 is sized and configured to receive guide 30 of an implant inserter, such as inserter 29 shown in FIG. 7. Channel 23 can be any size so long as guide 30 can be inserted therein and channel 23 allows for reciprocation of head 8 along the guide 30. For example, the diameter/thickness of channel 23 can be between about approximately 5 mm and about approximately 35 mm, preferably between about approximately 6 mm and about approximately 24 mm, and most preferably between about approximately 7 mm and about approximately 20 mm. When a guide 30 is positioned in channel 23, it may be desirable to provide a mechanism or means to retain the guide 30 in channel 23 during use. FIG. 4 shows a cross-sectional view of head 8 with an exemplary retaining member, ball plunger 25.

Head 8 may have one or more ball plungers 25. Ball plunger 25 may have a housing 28 containing spring 27 and ball bearing 31. The housing is configured to prevent ball bearing 31 from falling out of the housing while, at the same time, allowing a portion of ball bearing 31 to protrude from the housing into channel 23. Ball bearing 31 may also be held in the housing by being fixed to spring 27. Ball bearing 31 is permitted to move up and down in the housing in response to a force thereon, for example, by insertion of rod 30 into channel 23. Ball plunger 25 may be positioned in hole 33 (or an opening) of head 8. Plunger 25 can have external threads (not shown) to engage threads (not shown) in the perimeter of hole 33, thus keeping plunger 25 in place. Alternatively, plunger 25 may be connected to head 8 in various ways, including force fitting, welding, chemical or molecular bonding, or gluing. The position of plunger 25 in channel 23 or slot 23a (i.e., the extent to which plunger 25 extends into channel 23 or slot 23a) depends on the guide that is used and the size of the channel 23. It should be understood that ball bearing 31 can be any shape, such as for example a sphere, sloped triangular (FIG. 4A), hemi-spherical (FIG. 4B), straight sided, or triangle (FIG. 4C). The shape of ball bearing 31 should allow the guide 30 to move in and out of channel 23 when a predetermined force is applied to remove tool 1 from the guide 30. Once the guide 30 has passed the retaining member, the head 8 preferably is permitted to move along the guide 30. In an alternative embodiment, the retaining member may extend a portion of the length or the entire length of the channel 23. In another embodiment, the retaining member may be, for example, a screw, set screw, pin, bar or rod, which can be positioned into or on head 8 and/or channel 23. One of skill in the art can appreciate that other retaining mechanisms or means of holding or retaining a guide 30 in channel 23 is envisioned so long as the guide 30 can be moved in and out of channel 23 and head 8 can move along the guide 30. Head 8 may be also be configured such that the retaining member can be adjusted (i.e., the holding member can be positioned anywhere within channel 23; the holding member can be adjusted to extend into channel 23 to various extents).

FIG. 4E illustrates an alternative holding member, component 40 having finger 42. It should be understood by those of skill in the art that component 40 can be used by itself or in conjunction with other holding members such as, for example, ball plungers 25. A portion of component 40 (not shown) can be operatively connected to shaft 4. A pin, screw, set screw, bar or rod 44 can be positioned through an opening 46 in head 8, though an opening (not shown) in component 40, and/or into a receiving portion (not shown) within head 8. It should be understood by those of skill in the art that any construction that allows component 40 to rotate or pivot within the head 8 is envisioned, including rotation or pivot within slot 48 as shaft 4 is rotated or pivoted. Alternatively, the component 40 may be designed so that the finger 42 moves independently of shaft 4. Component 40 can be configured such that finger 42 can be extended (partially or entirely) into channel 23 and/or retracted (partially or entirely) into slot 48 and out of channel 23. For example, component 40 can be designed so that it moves straight up and down, into and out of channel 23 through slot 48. However, even in this up and down configuration, component 40 can be attached to shaft 4.

To position the head 8 on a guide 30, such as longitudinal member 30a, component 40 (by itself, by rotating/pivoting component 40 along with shaft 4, or by moving component 40 straight up and down with shaft 4) can be rotated, pivoted, or moved straight down into slot 48. Finger 42 can be entirely positioned within the slot 48 so that no portion of finger 42 is within the channel 23. However, finger 42 only needs to be positioned as far into slot 48 as necessary so that guide 30 can enter channel 23. It is desirable that when head 8 is positioned on a guide 30, such as longitudinal member 30a, and reciprocated thereon, the component 40 does not rotate or pivot to such a degree that the head 8 becomes disengage from the guide 30.

Moreover, head 8 can have multiple holes 33 (or opening) to allow an operator to adjust the position of the holding member, such as plunger 25, in head 8. Also, head 8 and plunger 25 can be configured to allow an operator to adjust the extent to which ball bearing 31 extends into channel 23. It should be noted that any mechanism that can be used to temporarily retain a guide 30, such as longitudinal member 30a, in channel 23 is envisioned.

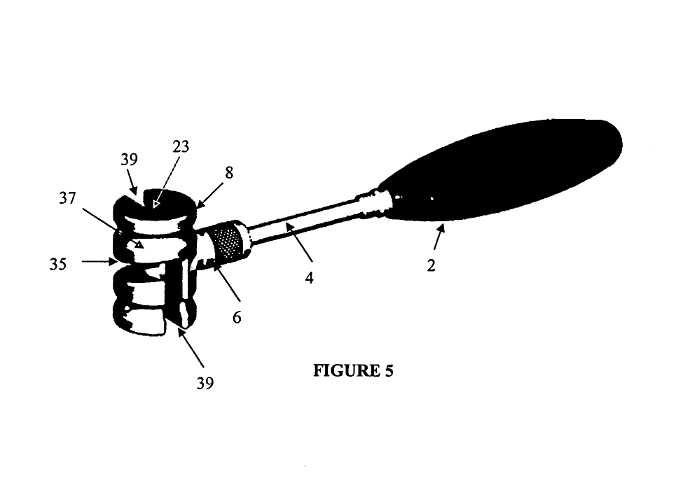

Turning now to FIG. 5, illustrated is an alternative embodiment of head 8. In this embodiment, a guide 30, such as longitudinal member 30a, of an implant inserter, such as inserter 29, can be twisted into channel 23. A slot 35 can extend between outer surface 37 of head 8 and communicate with and intersect channel 23. The axis of slot 35 may be transverse to, or more preferably oriented at a 90° angle relative to the longitudinal axis 9 of channel 23. It should be understood that any angle between about approximately 0° and about approximately 180° is also envisioned. Furthermore, channel 23 may include opposing side channels 39, which communicate with channel 23 and outer surface 37 of head 8, thereby forming paths for inserting the guide 30, in channel 23. The slot 35 in head 8 is positioned onto the guide 30 and then rotated through side channels 39 and into channel 23, such that the longitudinal axis of the guide 30 is aligned with the longitudinal axis 9 of channel 23. In another alternative embodiment, there can be a helical slot that extends from the outer surface of head 8 to channel 23.

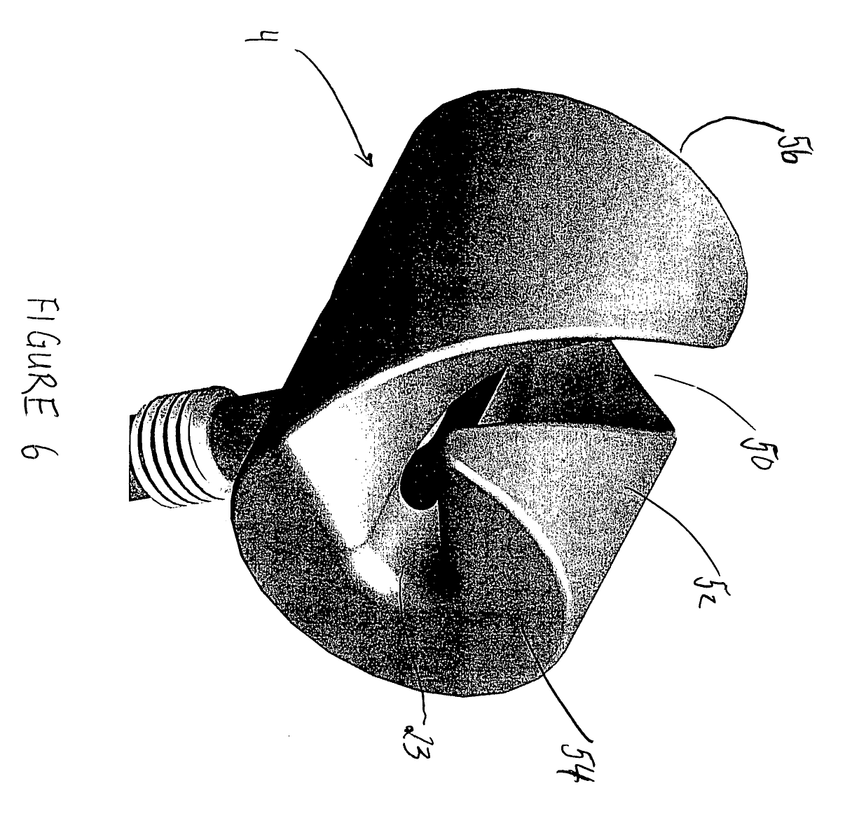

FIG. 6 shows another alternative embodiment of head 8. In this embodiment, head 8 can have a helical slot 50. Head 8 can be threaded, rotated or twisted onto a guide 30, such as longitudinal member 30a, of an implant inserter, such as inserter 29. The helical slot 50 can extend between outer surface 52 of head 8 and communicate with and intersect channel 23. Helical slot 50 can also intersect front face 54 and back face 56 of outer surface 52. As helical slot 50 in head 4 is advanced over guide 30, head 4 will thread, twist or rotate into channel 23, such that the longitudinal axis of guide 30 is aligned with the longitudinal axis 9 of channel 23.

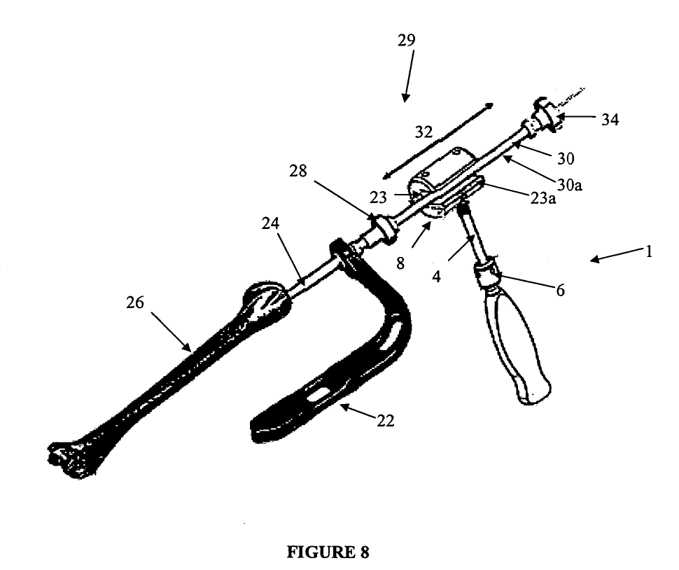

FIGS. 7 and 8 illustrate methods of using tool 1 with a implant inserter device, such as inserter 29, to insert, back-out, remove, position or reposition an implant, such as intramedullary rod 24, from bone 26. FIG. 7 shows the use of tool 1 to insert an implant, such as rod 24, into bone 26. To use tool 1 as shown in FIG. 7, head 8 is preferably fixed with respect to shaft 4 so that head 8 cannot pivot or rotate about shaft 4. A locking mechanism 7, such as locking member 6, can be used to fix head 8 relative to shaft 4. Head 8 is fixed by threading locking member 6 on threads 16 (FIG. 2) at distal end 18. Locking member 6 is tightened as described above so that it firmly engages head 8. It should be understood that any locking mechanism can be used to fix head 8 with respect to shaft 4 so long as head 8 does not pivot and/or rotate on shaft 4. In this configuration, the head 8 is in a “fixed position.”

In the fixed position, head 8 can be used to strike or hammer an implant, such as intramedullary rod 24, into bone 26. To assist in hammering rod 24 into bone 26, an implant inserter, such as inserter 29, may be provided. Tool 1, however, can be used to hammer the implant directly. Inserter 29 is attached to rod 24 and includes a mushroom-shaped head portion 28 and an aiming arm 22. In use, an operator and/or an assistant holds aiming arm 22 by hand and the operator and/or the assistant strikes portion 28 with head 8 of tool 1. By swinging tool 1 in direction 32a and striking portion 28, rod 24 is driven in bone 26. It should be understood that head 8 preferably may be fixed with respect to shaft 4 when tool 1 is used with an inserter 29 shown in FIG. 7 or with no inserter at all. It is also contemplated that head 8 may pivot with respect to shaft 4 when used with inserter 29 shown in FIG. 6 or without any inserter at all.

Alternatively, FIG. 8 illustrates the use of tool 1 to insert, back-out, remove, position or reposition an implant, such as rod 24, from bone 26. To use tool 1 as shown in FIG. 8, head 8 preferably should be permitted to pivot with respect to shaft 4. To accomplish this, the locking mechanism 7, such as locking member 6, is disengaged from head 8 by loosening member 6 from threads 16 and moving locking member 6 towards proximal end 14 of shaft 4. Locking member 6 may be kept stationary on shaft 4, so that it does not move during swinging of tool 1, by threading locking member 6 on threads 12 (FIG. 1) at proximal end 14. The locking member 6 is now spaced a distance from head 8 sufficient enough to allow head 8 to pivot with respect to shaft 4 in a direction shown by arrow 20 (FIG. 2). As discussed above, depending on how shaft 4 and head 8 are attached, head 8 may be able to rotate about shaft 4. In this configuration, the head 6 is in a “toggle position” and can act similar to a slaphammer or slide hammer.

In the toggle position, head 8 may be used with an implant inserter device, such as inserter 29, to hammer an implant, such as rod 24, into and/or out of bone 26. Inserter 29 is attached to rod 24 and includes an aiming arm 22, a distal mushroom-shaped head portion 28, a guide 30 (e.g., a longitudinal member 30a,), and a proximal mushroom-shaped head portion 34. One of ordinary skill in the art would appreciate that guide 30 may be any shape, including round, oval, square, rectangular, or other polygon, and can have any thickness so long as head 8 can fit over guide 30 and move therealong. Head 8 of tool 1 is positioned on guide 30 by moving slot 23a over guide 30 until guide 30 enters channel 23. Guide 30 may be retained in channel 23 using a holding member as described above. To insert rod 24 into bone 26, an operator and/or an assistant holds arm 22 by hand and the operator and/or the assistant reciprocates head 8 on guide 30 in direction 32, repeatedly striking portion 28 with head 8 of tool 1. In this manner, rod 24 is driven in bone 26. To back-out or remove rod 24 from bone 26 or reduce a fracture (i.e., pull two bone fragment together) using rod 24, or reposition rod 24 in the bone, the operator and/or the assistant reciprocates head 8 on guide 30 in direction 32, repeatedly striking portion 34 with head 8 of tool 1.

The tool of FIG. 5 can be used by using the same reciprocating motion as described above. Also as discussed above, a locking mechanism, such as locking member 6, can be used to fix head 8 relative to shaft 4 and can be positioned to allow head 8 to toggle with respect to shaft 4. Only the method of positioning head 8 onto guide 30 differs. Slot 35 is positioned onto the guide 30 until the guide 30 intersects channel 23. At this point, the longitudinal axis of the guide 30 is at an angle with the longitudinal axis of channel 23. Head 8 is then rotated such that the guide 30 passes through opposing side channels 39 and into channel 23. At this point, the longitudinal axis of the guide 30 aligns with the longitudinal axis 9 of channel 23. The same mechanism and structures used to temporarily retain the guide 30 in head 8 of FIGS. 1 and 2, such as for example plunger 25, can be used to temporarily retain the guide 30 in the embodiment of head 8 illustrated in FIG. 5. In yet another embodiment, shown in FIG. 6, a guide 30, such as longitudinal member 30a, can be positioned in a helical slot 50. As the helical slot 50 is advanced over the guide 30, the head 8 threads, rotates or twists such that guide 30 is positioned in channel 23. At this point, the longitudinal axis of the guide 30 aligns with the longitudinal axis 9 of channel 23.

While the foregoing description and drawings represent the preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

1. A device for use with a bone implant comprising:
an impacting body;
a channel through the impacting body, the channel having a longitudinal axis;
a shaft having a proximal end and distal end, the distal end of the shaft being operatively connected to the impacting body such that the shaft is permitted to pivot with respect to the impacting body; and
a locking mechanism having at least two operating positions, a first position wherein the locking mechanism permits the shaft to pivot with respect to the impacting body and a second position wherein the locking mechanism prevents the shaft from pivoting with respect to the impacting body.

2. The device of claim 1, wherein the impacting body has an axis and the longitudinal axis of the channel is parallel to the axis of the impacting body.

3. The device of claim 2, wherein the impacting body has an outer surface and the channel intersects the outer surface of the impacting body.

4. The device of claim 3, wherein the impacting body comprises a slot communicating with the channel and the outer surface of the impacting body.

5. The device of claim 4, wherein the slot is at an angle relative to the longitudinal axis of the channel.

6. The device of claim 4, wherein the slot is parallel to the channel.

7. The device of claim 1, further comprising a handle.

8. The device of claim 1, wherein at least a portion of the shaft is threaded.

9. The device of claim 8, wherein the shaft has threads proximate the impacting body and threads proximate the proximal end of the shaft and an unthreaded portion tnerebetween.

10. The device of claim 9, wherein the locking mechanism comprises a locking member associated with the shaft, the locking member moveable to a fixed position on the shaft such that the locking member is fixed with respect to the shaft and the shaft can pivot with respect to the impacting body.

11 The device of claim 8, wherein the locking mechanism has an internal thread that engages the thread portion of the shaft.

12. The device of claim 1, wherein the locking mechanism in the first position disengages from the impacting body and in the second position engages the impacting body.

13. The device of claim 1, wherein the locking mechanism comprises means for tightening the locking mechanism on the shaft.

14. The device of claim 13, wherein the means for tightening comprises a portion for receiving a tool.

15. A device for use with a bone implant comprising:
a head having an outer surface;
a channel through the head, the channel having a longitudinal axis;
a slot intersecting the channel and the outer surface of the head,
a shaft having a proximal end and distal end, the distal end of the shaft is operatively connected to the head; and
a locking mechanism having at least two states, a first state wherein the locking mechanism permits the shaft to pivot with respect to the head and a second state wherein the locking mechanism prevents the shaft from pivoting with respect to the head.

16. The device of claim 15, wherein the slot is non-parallel to the longitudinal axis of the channel.

17. The device of claim 15, wherein the slot is parallel to the channel.

18. The device of claim 15, wherein the head has an axis and the longitudinal axis of the channel is parallel to the axis of the head.

19. The device of claim 15, further comprising side channels, the side channels communicating with the channel, the outer surface of the head and the slot.

20. The device of claim 15, further comprising a handle.

21. The device of claim 15, wherein the locking mechanism in the first state disengages from the head and in the second state engages the head.

22. The device of claim 15, wherein the locking mechanism comprises means for tightening the locking mechanism on the shaft.

23. The device of claim 15, wherein the slot is a helical slot intersecting the channel and the outer surface of the head.

24. The device of claim 15, wherein at least a portion of the distal end of the shaft comprises threads.

25. The device of claim 24, wherein the locking mechanism is configured and designed to engage the threads on at least a portion of the distal end of the shaft, the locking mechanism contacting the head when engaging the distal threads to prevent movement of the head relative to the shaft.

26. The device of claim 15, wherein at least a portion of the proximal end of the shaft comprises threads.

27. The device of claim 26, wherein the locking mechanism is configured and designed to engage the threads on the at least a portion of the proximal end of the shaft, the locking mechanism positioned a distance from the head when engaging the proximal threads to permit movement of the head relative to the shaft.

28. A device for impacting, positioning and removing a bone implant, comprising:
a head;
a channel through the head;
a shaft having at least a threaded portion, a proximal end and a distal end, the distal end of the shaft operatively connected to the head to permit pivoting of the head with respect to the shaft; and
a locking member having an internally threaded portion, the locking member moveable from a first position wherein the locking member permits the shaft to pivot with respect to the head to a second position where the threaded portion of the locking member engages the threaded portion of the shaft to position the locking member in contact with the head and prevent the shaft from pivoting with respect to the head.

29. The device of claim 28, wherein the locking member in the first position disengages from the head.

30. The device of claim 28, further comprises means for retaining a guide in the channel.

31. The device of claim 28, wherein the means for retaining is a plunger, the plunger comprising a spring and bearing in the head, the bearing being capable of movement in the channel to retain the guide in the channel.

32. The device of claim 28, wherein the means for retaining is a finger, the finger being moveable within the channel.

33. The device of claim 28, wherein the means for retaining defines a first diameter in the channel for receiving the guide, the means for retaining being adjustable such that the means for retaining defines a second diameter in the channel for receiving the guide.

34. A method of moving an implant relative to bone comprising:
attaching an inserter having a guide to the implant;
providing a device comprising:
an impacting body;
a channel through the impacting body;
a shaft having a proximal end and distal end, the distal end of the shaft is operatively connected to the impacting body; and
a locking mechanism operatively associated with the shaft, wherein the locking mechanism has at least two states, a first state wherein the locking mechanism permits the shaft to pivot with respect to the impacting body and a second state wherein the locking mechanism prevents the shaft from pivoting with respect to the impacting body;
positioning the impacting body on the guide of the inserter; and
reciprocating the impacting body on the guide of the inserter.

35. The method of claim 34, further comprising the step of:
placing the locking mechanism in the second state.

36. The method of claim 34, further comprising the step of:
placing the locking mechanism in the first state.

37. The method of claim 36, wherein the guide comprises a distal end proximate the implant, a proximal end opposite the distal end, and an enlarged portion proximate the distal end of the guide.

38. The method of claim 37, further comprising the step of:
striking the enlarged portion with the impacting body, thereby moving the implant.

39. The method of claim 36, wherein the guide comprises an increased diameter portion proximate the proximal end of the guide.

40. The method of claim 39, further comprising the step of:
striking the increased diameter portion with the impacting body to move the implant.

41. The method of claim 34, wherein the device further comprises a slot intersecting the channel and the outer surface of the head.

42. The method of claim 41, further comprising the step of:
moving the slot onto the guide such that the guide enters the channel.

43. The method of claim 42, wherein the slot is non-parallel to the longitudinal axis of the channel.

44. The method of claim 43, wherein the head of the device further comprises side channels, the side channels communicating with the channel and the outer surface of the head, thereby forming a path.

45. The method of claim 44, further comprising the steps of:
rotating the device such that the guide passes through the side channels and the longitudinal axis of the channel is aligned with the longitudinal axis of the guide; and
reciprocating the head on the inserter.

46. The method of claim 41, wherein the slot is a helical slot

47. The method of claim 46, further comprising the step of:
attaching an inserter to the implant, the inserter having a guide with a longitudinal axis.

48. The method of claim 47, further comprising the steps of:
moving the slot onto the guide;
turning the device until the guide enters the channel and the longitudinal axis of the channel is aligned with the longitudinal axis of the guide; and
reciprocating the head on the guide.
